(19) United States
(12) Patent Application Publication
(10) Pub. No.: US 2005/0165429 A1
(43) Pub. Date: Jul. 28, 2005

(54) Surgical clamp possessing a combined parallel and scissor style clamp head

(76) Inventors: Peter Douglas, New Milford, NJ (US); Scott G. Corey, Somerville, MA (US); Randolph Chitwood, Greenville, NC (US); Stephen Evans, Westford, MA (US); Darrell Hartwick, Newton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/764,102

(22) Filed: Jan. 23, 2004

(51) Int. Cl. A61B 17/08

(52) U.S. Cl. 606/157

(57) ABSTRACT

A surgical clamp includes a clamp head, and a first jaw and a second jaw, each mounted to the clamp head for movement toward and away from each other. The clamp also includes an operative mechanism coupled to the proximal end of at least one of the first jaw and the second jaw, and an actuating structure connected to the operative mechanism for imparting a parallel opening movement to at least one of the first jaw and the second jaw from a first position to a second position where the first jaw and the second jaw are spaced apart and parallel to one another, the actuating structure selectively imparting further scissors-like opening movement to at least one of the first jaw and the second jaw from the second position to a third position where the first jaw and the second jaw are spaced apart a distance greater than that of the second position.

### BACKGROUND OF THE INVENTION

1. Field of the Invention

The present invention relates to devices for occluding hollow body vessels, and in particular, relates to a surgical clamp having a jaw structure that operates to close and open in a parallel fashion and also in a scissor-style fashion. This structure is especially beneficial when the clamp is used to occlude larger-sized hollow body vessels, such as the aorta. Furthermore, the invention is directed to the implementation of body vessel occluding procedures through the use of the inventive surgical clamp structures. Moreover, a specific application of the present invention is directed to the utilization of the surgical clamp incorporating a clamp head structure in an endoscopic procedure wherein the clamp is incorporated in less invasive medical devices, such as endoscopes, and provides for the atraumatic occlusion of the hollow body vessels, such as, in particular, although not in any manner limited to, isolating heart and coronary blood vessels from the flow of blood from the remaining constituents of the arterial system of a patient.

In general, surgical clamps or hollow body vessel occluding devices of the type that are widely employed in the medical and surgical technology, utilize a scissors type motion, wherein the jaws of the clamp open and close in a generally subtending relationship. In this manner, when a body vessel to be occluded is located between the jaws, the cooperating jaws apply a pressure to the vessel in a kind of scissors clamping action. This clamping action has a traumatic effect on the vessel being occluded, as the portion of the vessel located proximate the jaw hinge is compressed prior to the portion of the vessel distal the jaw hinge. This often results in the body vessel being overcompressed and traumatized at the location near the jaw hinge as the tissue at that location continues to be compressed while the distal ends of the jaws move together. Conversely, the portion of the body or blood vessel located distal to the jaw hinge may not be compressed sufficiently to fully occlude the vessel, resulting in blood flowing through the vessel, albeit at a reduced rate.

Occluding devices for hollow body vessels in the form of surgical clamps are known to include vessel clamping jaws that open and close in a generally parallel motion between each other. This type of clamp is somewhat less traumatic in the sense that the facing contact surfaces of the clamping jaws engage the surface of the body vessel in a uniform manner so as to distribute the clamping force or pressure evenly, thereby ensuring complete vessel occlusion while reducing compressive stresses in the body vessel and any resulting traumatic effects. Although the foregoing parallel motion of the clamp jaws is adapted to reduce compressive stresses or localized excessive forces acting on the hollow vessel that is being occluded, present clamp head structures of that type do not permit the occlusion of larger sized vessels, for instance, such as the aorta of a patient, without necessitating an increase in the overall dimensions of the clamp head to facilitate a wider spring between the clamps. Such a design renders the clamp head unsuitable for use in endoscopic procedures and decreases its utility in minimally invasive surgery.

2. Discussion of the Prior Art

Body vessel occluding clamps that employ various types of clamping jaws and clamping configurations in their deployment, are well known in the medical and surgical technology. Maleki, et al., U.S. Pat. No. 5,626,607, discloses a surgical clamp assembly for the occluding of hollow body vessels and methods of use thereof wherein various types of clamp configurations have actuating structures causing the jaws to open and close in generally parallel motion relative to each other so as to thereby reduce localized excessive pressures or forces acting on the body vessel being occluded and imparting generally atraumatic clamping action to the body vessel. Maleki also provides clamps that include jaws that open and close in a scissors-type motion. Maleki, however, does not disclose a clamp that includes a composite parallel and scissors-type actuation of the clamp jaws.

Fogarty, et al., U.S. Pat. No. 4,821,719 and Patent Publication Nos. 2002/0049470 and 2002/0111650, each disclose surgical clamps that provide replaceable and elastomeric pads for the purpose of dispersing the forces across larger surface areas of a body vessel being occluded by the clamp jaws and to reduce the traumatic effect on the body vessel. These particular clamp head structures as disclosed therein provide for clamp jaws actuatable in a scissors-type movement, which does not permit for the parallel clamping motion between the clamp jaws, or any combined parallel and scissors-type clamping movement to accommodate larger-sized body vessels without the necessity of increasing the size of the clamp head mounting jaws.

### SUMMARY OF THE INVENTION

Although Maleki provides for parallel motion between the jaws of a surgical clamp upon opening and closing thereof, the latter contrary to the present invention, is not designed for a compound jaw motion. That is, one that combines a parallel opening and closing movement between the jaws of the clamp head in engaging a body vessel and a successive scissors-type movement enabling a larger opening to be effected between the jaws of the clamp to accommodate vessels that might not otherwise be accommodated within the opening provided by jaws having a parallel-only type motion. In effect, contrary to the current state-of-the-art, the present invention facilitates the construction of small sized clamp heads to be employed in minimally invasive surgery, such as in endoscopy, that are capable of occluding comparatively larger body vessels without the necessity of having to increase the size of the jaw head mounting the clamping jaws.

Ordinarily, the surgical clamps presently being marketed implement a scissors-type motion during closing of the jaws of the surgical clamps so as to exert a pressure on the body vessel at the proximal end of the clamp jaw which is much higher than that encountered at the distal or free end of the jaws, tending the body vessel being occluded to be subjected to an overcompression at the proximal end, whereas the distal or free end of the clamp jaw may not fully occlude the body vessel located between the clamp jaws. As having been investigated in the medical technology, a parallel or uniform motion between the clamp jaws will distribute the clamping action or occlusion more uniformly than would a scissors-type motion between the jaws.

Although the prior art, as represented in Malecki, provides for the parallel motion of the clamp jaws, the concept disclosed therein is inadequate to enable small sized clamp heads, which are to be passed through an endoscope or trocar, so as to be adequate to effect, by way of non-limiting example, aortic clamping while the patient may be on a cardio-pulmonary bypass (CPB), such as on a heart-lung machine. Thus, during a stopped heart procedure, a CPB system is connected to the circulatory system to provide oxygenated blood to the patient. At this point the ascending aorta is clamped or occluded, and the surgeon delivers cardioplegia into the coronary arteries to arrest the heart. Once the aortic clamp has been applied, the heart and lungs are isolated from the rest of the circulatory system and the CPB system takes over the pumping and oxygenating functions of those organs. During this time, the clamp prevents blood from entering the heart through the coronary arteries or an incompetent aortic valve. Once the surgical procedure is completed, the surgeon removes the clamp to allow warm blood into the coronary arteries which re-establishes cardiac function.

Thus, a primary difference between the inventive surgical clamp structure and the standard clamps is the capability of initially receiving the vessel that is to be occluded with the clamp jaws in a wider scissors-type opening, and then during closing movement imparting a final closing phase in a parallel jaw motion. This combined or composite jaw motion enables the surgical clamp to accommodate, for example, the larger-sized aorta for occlusion of the latter, without having to increase the dimensions of the clamp head. By combining a scissors-type motion for the clamp jaws to initially grasp the vessel, and thereafter converting the scissor-type movement to a parallel action, the tissue of the body vessel clamped between the jaws is substantially, proportionately compressed to achieve occlusion. The addition of the scissors-type motion to the parallel motion into the combined actuation of the jaws enables the use of a small clamp head that can be used in minimally invasive surgery, such as in conjunction with an endoscope or laparoscope.

In accordance with an embodiment of the invention, a clamp head is provided in which a combined scissors-type and parallel motion between paired clamp jaws is implemented by means of a suitable linkage and pin system, imparting articulation in which at least one of the jaws is maintained in a straight or essentially unmoved position, whereas the mating jaw will open initially in a parallel motion relative thereto and thereafter open further in a scissors-type motion and effecting a reverse order in movement for the occlusion of a body vessel between the jaws.

Pursuant to another embodiment of the invention, the linkage and pin mechanism may be actuated so as to permit both of the jaws to initially open or finally close in a parallel motion relative to each other and thereafter both jaws angled outwardly to further open or to close in a scissors-type motion so as to be able to accommodate larger-sized body vessels in the absence of increasing the size of the clamp head mounting the jaws.

Pursuant to a still further embodiment of the invention, the motion between the jaws in essentially initial or final parallel clamping action and thereafter in a wider opening or closing scissors-type movement, in which at least one of the jaws or both jaws are actuated or pivoted relative to the other jaws in combined parallel and scissors-type increments is implemented by means of an angled slot configuration in the clamp head by which the jaws are hingedly connected.

Pursuant to the invention, there are disclosed novel methods of utilizing the jaw heads for surgical clamps, mounting paired jaws articulated in combined parallel and scissors-type motions for the occlusion of hollow body vessels, as disclosed herein.

### BRIEF DESCRIPTION OF THE DRAWINGS

Reference may now be made to the following detailed description of preferred embodiments of the invention, taken in conjunction with the accompanying drawings; in which:

FIG. 1 illustrates a side view of a first embodiment of a clamp head for a surgical clamp pursuant to the invention, shown in a fully closed clamping position of the clamp jaws;

FIG. 2 illustrates a side view of the surgical clamp head of FIG. 1 in a partially opened position representing the parallel displacement of the clamp jaws;

FIG. 3 illustrates a side view of the surgical clamp head of FIG. 1 in the fully opened position;

FIG. 4 illustrates a side view of a perspective end and side view of the surgical clamp head of FIG. 1, shown in the fully opened position thereof.

FIG. 5 illustrates a top plan view of the surgical clamp head;

FIG. 6 illustrates a side view of a modified embodiment of the surgical clamp head, shown in the fully opened position thereof;

FIG. 7 illustrates a side view of further embodiment of the surgical clamp head, shown in the fully opened position thereof;

FIG. 8 illustrates a perspective exploded side view of the surgical clamp head of FIG. 7;

FIG. 9 illustrates a side view of another embodiment of the surgical clamp head, shown in the opened position thereof; and

FIG. 10 illustrates a perspective exploded side view of the surgical clamp head of FIG. 9.

### DETAILED DESCRIPTION OF THE PREFERRED EMBODIMENTS

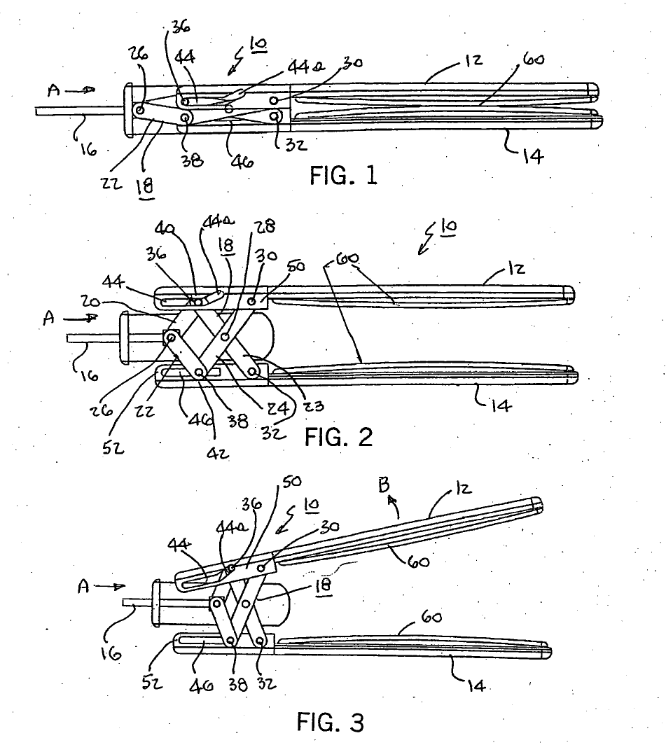

Referring now in more specific detail to the embodiment disclosed in FIGS. 1 to 4, there is illustrated the components of a surgical clamp, such as a surgical clamp head 10 mounting a pair of clamp jaws 12, 14, which is configured for effectuating a parallel clamping motion between the paired jaws 12, 14 of the clamp, as is depicted in FIG. 2, combined with a scissors-type further opening movement, as depicted in FIG. 3, to accommodate hollow body vessels, such as the aorta of a patient or the like. The surgical clamp head 10 can be actuated or operated by means of a suitable cable 16, and is preferably of a size that can be accommodated in an endoscope or laparoscope (not shown), for example, a 10 mm trocar. Clamp head 10 includes an actuating structure or linkage mechanism 18 that is connected to cable 16 and includes a plurality of pivotable links 20, 22, 23 and 24 that are interconnected into a parallel folding linkage system by means of pivot pins 26, 28, 30 and 32. The linkages have guide pins 36, 38 at opposite ends 40, 42 that are slidable in parallel slots 44, 46. Slot 44 is formed in a first plate 50 that is connected to upper or first jaw 12. Slot 44 includes a slot segment 44a that extends at an angled orientation away from slot 44 at one end thereof. Slot 46 is formed in a second head plate 52 that is connected to lower or second jaw 14.

Jaws 12, 14 are shown in the closed position in FIG. 1, in the intermediate, parallel opened position in FIG. 2, and in the fully open position in FIG. 3. Cable 16 is actuated in a direction shown as arrow A in FIGS. 1, linkage mechanism 18 and, more specifically, pivot 26, is moved in the same direction as cable 16, thereby spreading links 20, 22, 23 and 24 and moving guide pins 36, 38 within slots 44, 46. Jaws 12, 14 are moved apart from one another in a generally parallel orientation from a fully closed position, shown in FIG. 1, to the intermediate position of FIG. 2, when guide pin 36 moves within the straight segment of slot 44. In the intermediate position, a user can position a body vessel between jaws 12, 14 and occlude the vessel by pulling of cable 16 in a direction opposite to the direction of arrow A. In such a case, the linkages will cause the guide pins 36, 38 to slide back within the parallel portions of the upper and lower slots 44, 46. This type of parallel opening and closing motion between the upper and lower jaws 12, 14 provides an even distribution between clamping forces or compressive pressures on the occluded vessel from both the proximate and distal ends of the jaws.

In the event that the clamp needs to accommodate a vessel having a diameter that is larger than a diameter accommodated in a parallel configuration, as shown in FIG. 3, the jaws 12, 14 may be further opened by a more extensive cable actuation of the linkage mechanism 18 that causes the guide pin 36 in the slot 44 of the upper jaw 12 to move into the upwardly extending angled slot portion 44a, while the guide pin 38 in the slot 46 moves to the end of the latter, thereby imparting an upward pivoting motion B to the upper jaw creating a scissors-type angular opening between the upper and lower jaws 12, 14. Thereafter, during the closing of the jaws 12, 14 by the actuation of the cable 16 and linkage mechanism in a reverse to arrow A, the initial compression of the larger body vessel will be via a scissor-type closing motion between the upper and lower jaws, but prior to the full closing thereof, upon assuming the partially open parallel position of the upper and lower jaws (FIG. 2), the full clamping closure is then implemented in a parallel motion between the jaws. Inasmuch as the guide pin 38 in the upper slot 44 is again guided into the portion of the slot extending in parallel with the lower slot 46, this causes the occlusion of the body vessel to be completed in a generally parallel final closing phase of motion between the upper and lower clamp jaws 12, 14.

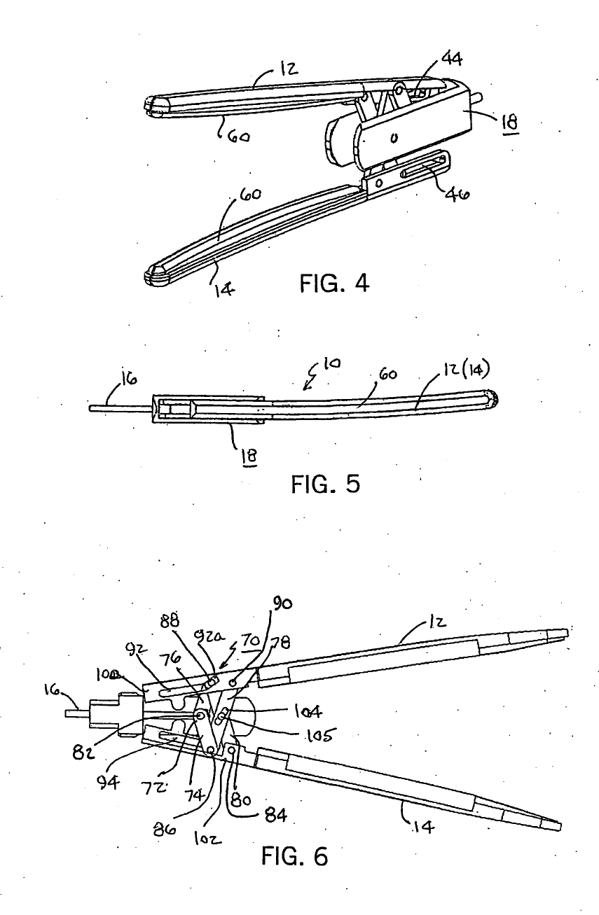

As depicted in the perspective view of FIG. 4, the pivoting motion of jaws 12, 14 provided by linkage mechanism 18 is imparted primarily to upper jaw 12, with lower jaw 12 remaining in a basically fixed position to effect the combined parallel and scissors-type clamping action during the opening and closing of the jaws.

It should be understood that one skilled in the art can envision other ways in which to impart the parallel- to scissors-type opening and closing motion of jaws 12, 14. For example, the angled portion 44a of slot 44 could be on the proximal end of clamp 10 rather than the more distal location as shown in FIGS. 1-3. In such an embodiment, angled slot portion 44a would extend downwardly rather than upwardly as shown in the figures, and to open jaws 12, 14, cable 16 would pull linkage assembly 18 rather than push linkage assembly 18 as shown in the figures.

To avoid any injury to the tissue of the occluded vessel, suitable elastomeric or resilient pads 60 may be mounted on or in the facing surfaces of the upper and lower jaws 12, 14. Such pads may be of a relatively soft fabric or plastic material possessing suitable surface characteristics that can further distribute the forces along the surfaces of the occluded body vessel, and thereby minimize injury to the body vessel by reducing trauma caused by the clamping action of the jaws.

As shown in the top plan view of FIG. 5, clamp head 10 may be of a configuration in which jaws 12, 14 are imparted with a slight lateral or sideways curvature along their axial lengths to accommodate the curvature of body vessels. It is also possible that the jaws can be straight, as shown in FIGS. 1 to 4, or be imparted with other curvatures along their axial lengths.

Pursuant to a non-limiting exemplary embodiment of the invention representative of surgical clamp head 10, the working length of each of jaws 12, 14 (in effect, the length of the jaw with jaw pads 60 thereon) is preferably approximately 65-75 mm. Jaws 12, 14 themselves are approximately 5-7 mm wide, with a lengthwise curvature of a radius of 150-225 mm. Jaws 12, 14 provide for a parallel motion for the initial opening or final closure of about 10-12 mm, with the remainder of the opening motion being of the scissors-type. The activation stroke of cable 16 along the direction A is approximately 10 to 20 mm in length. The entire surgical clamp head 10 is dimensioned to fit through a 10 mm diameter opening (i.e., such as a trocar), and the jaws should open sufficiently to therebetween engage and accommodate up to a 40 mm diameter vessel, such as the aorta of a patient.

In the following described embodiments, components of the surgical clamp head, which are similar to or identical with those described in connection with FIGS. 1-5, are identified with the same reference numerals.

In a modification of the surgical clamp, as shown in FIG. 6, a still further increase in the opening size between jaws 12, 14 of a clamp head 70 may be obtained by using a linkage mechanism 72 that provides a dual scissor and parallel open position. In this embodiment, plate structure 100 of upper jaw 12 has a slot 92 formed therein that, like the slot in the first embodiment, extends to an angled portion 92a, and a lower structure 102 of lower jaw 14 has a slot 94 formed therein that is generally parallel to slot 92. Link elements 74, 76, 78 and 80 are connected by pins 82, 84, 86, 88, 90, whereby in the fully opened position shown in FIG. 6, a guide pin 88 in the upper jaw rides within upwardly angled slot portion 92a, and guide pin 86 rides within slot 94. In addition, a slot 104 in link element 78 is configured to receive a guide pin 105 such that lower jaw 14 moves away from the upper jaw 12 at an angle into the fully opened jaw position when a force is applied to cable 16. During closing, the reverse motion of the pins into the parallel portions of the slots for both the upper and lower jaws will, again, cause the jaws to assume a parallel relationship prior to full closing thereof, and thereafter a final parallel movement occluding the body vessel which is clamped therebetween.

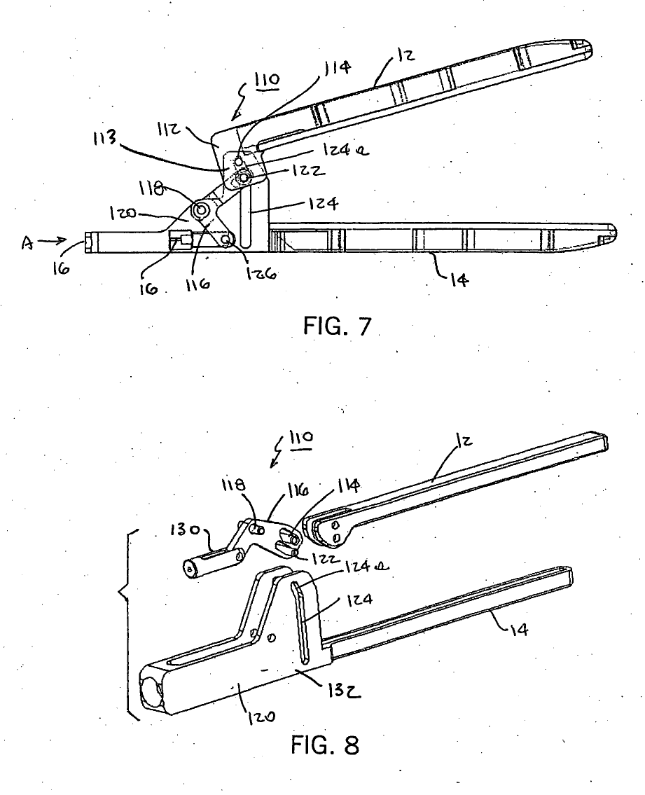

In another preferred embodiment, a clamp head 110 shown in FIG. 7 includes upper and lower jaws 12, 14, a plate 113, an L-shaped hinge 116, and an actuating connector 130 attached to hinge 116 via a lower pin 126. Hinge 116 is connected to plate 113 by means of a guide pin 122 and to a plate 112 formed at the proximal end of jaw 12 by means of a pin 114. Hinge 116 is also connected to a plate 120 formed at the proximal end of jaw 14 at a pivot point 118 and via guide pin 122 at an distal end of hinge 116. Guide pin 122 is slidable in a vertical slot 124 formed in plate 120. Vertical slot 124 includes an angled slot 124a, which extends proximally from vertical slot 124. FIG. 8 depicts an exploded view of the components of the clamp illustrated in FIG. 7, omitting plate 113 for clarity. Actuating connector 130 is connected to cable 16, and is arranged within a plate-like housing 132 attached to lower jaw 14.

Upon actuation of cable 16 in direction A, lower pin 126 moves in the same direction. Because hinge 116 is fixed at pivot point 118, movement in the direction of arrow A, causes pin 122 to move vertically within vertical slot 124 and upper and lower jaws 12, 14 move from a closed configuration to an open, parallel configuration. When pin 122 is displaced such that the pin 122 moves into angled slot portion 124a, the distal end of upper jaw 12 pivots outwardly relative to the distal end of jaw 14 in a scissors-type angular motion so as to accommodate a larger hollow body vessel between the jaws 12, 14. Again, as in the previous embodiments, the scissors-type opening or motion between upper and lower jaws 12, 14 is effected in the furthermost opening movement between the jaws, whereas during closing of the jaws, as the pin 122 in the angled portion 124a of the slot 124 moves downwardly into the vertical portion of slot 124, upper jaw 12 assumes a parallel orientation relative to lower jaw 14 and then a parallel closing motion as guide pin 122 moves down vertical slot 124. In this way, jaws 12, 14 clamp in the desired parallel configuration to occlude a body vessel disposed between jaws 12, 14.

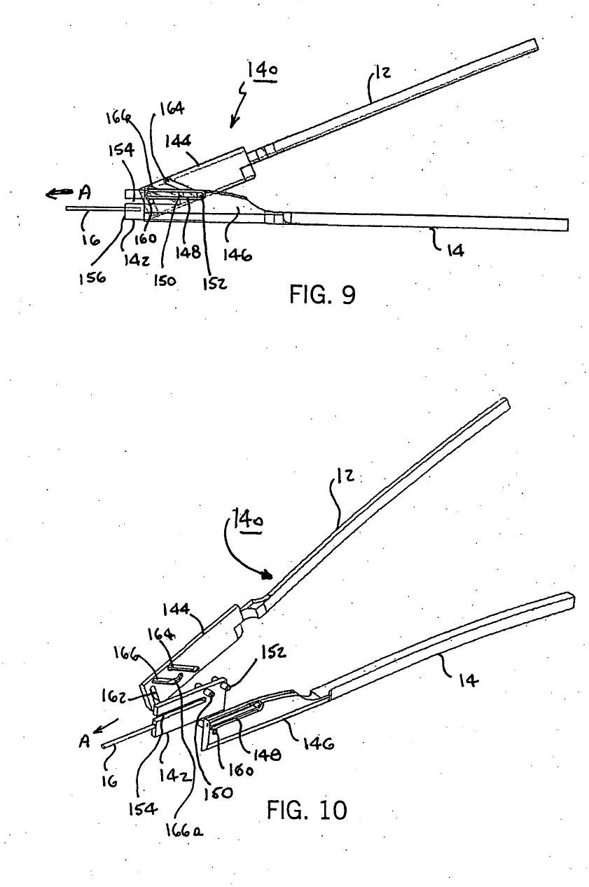

Referring to another embodiment, illustrated in FIGS. 9 and 10, a surgical clamp head 140 includes upper jaw 12, lower jaw 14 and a plate-shaped sliding carriage 142, which is adapted to be activated by cable 16 when cable 16 is displaced along the direction of arrow A. Jaws 12 and 14 are, respectively, provided with plate structures 144, 146 at their proximal ends flanking sliding carriage 142. Lower jaw 14 is provided with a linear elongated slot 148, which is essentially oriented in parallel with the axial extent of lower jaw 14. Sliding carriage 142 is equipped with a pair of spaced pins 150, 152 projecting orthogonally from both sides thereof. Pins 150, 152 are located above and distal of a slot 154 formed in carriage 142. Slot 154 is open-ended toward the proximal end 156 of sliding carriage 142 and is located above the point of attachment of cable 16 to carriage 142. Moreover, slot 154 in sliding carriage 142 is oriented in parallel with slot 148 of lower jaw 14. Pins 150, 152 on the side facing jaw 14 are adapted to ride within slot 148 formed in lower jaw 14, thereby causing jaw 14 to remain in its position. Lower jaw 14 can also be fixed to a suitable jaw housing (not shown).

Lower jaw 14 includes a pin 160 located below the rear end of slot 148. Pin 160 is of a length adapted to pass into and through slot 154 formed in sliding carriage 142 and also to extend further into a vertical slot 162 formed in the proximal end of plate structure 144 of upper jaw 12. This arrangement prevents upper jaw 12 from moving axially relative to lower jaw 14. Additionally, upper jaw 12 is provided with a first slot 164 and a second slot 166 formed in plate structure 144. First slot 164 is a straight slot extending at a specified angle relative to the axial extent of jaw 12, whereas second slot 166 is generally formed parallel with first slot 164, but has a curved portion 166a toward the lower end thereof. When assembled as shown in FIG. 9, the two sliding carriage pins 150, 152 ride within slots 164, 166.

Accordingly, prior to actuation, when jaws 12, 14 are in a closed position, pins 150, 152 are in the rearward- or proximal-most position within slot 148 of plate 146, and the upper position within slot 166 and 164, respectively. Upon actuation of cable 16 in the direction opposite to that shown as arrow A in FIGS. 9 and 10, upper jaw 12 commences with an opening motion in parallel with lower jaw 14 because slots 164, 166 are at identical angles with each other. When second pin 154 enters into lower curved portion 166a of second slot 166 formed in plate 144, however, the leading portion of upper jaw 12 swings upwardly so as to produce a scissors-type opening motion between jaws 12 and 14. In this way, jaws 12, 14 can engage larger body vessels, such as the aorta of a patient. When sliding carriage 142 is moved in the direction indicated by arrow A, pin 150 draws upper jaw 12 back into a parallel relationship with jaw 14 as pin 150 moves from slot portion 166a to slot 166. At this point, the jaws move in a parallel motion relative to each other during the final stage of being clamped into their mutually closed position.

In essence, in various of the embodiments, one of the jaws, such as the lower jaw 14, may be relatively immoveable or fixed with regard to the respective clamp head structure, and applicable actuation mechanism (e.g., linkages, slots and pivot mechanisms) are adapted to displace upper jaw 12 relative to lower jaw 14 in an initially essentially parallel opening, and to thereafter increase the opening between jaws 12, 14 in a scissors-type movement so as to be able to accommodate larger body vessels therebetween.

Alternatively, as indicated, the clamp head structures may also be such as to enable both jaws 12, 14 to move from an initial parallel opening motion into a scissors-type opening therebetween in order to accommodate even larger body vessels. The unique novel slidable and rotatable connections between the upper and lower jaws of the surgical clamp head end facilitates the occlusion of large body vessels, while maintaining the dimensions of the clamp as small as possible so as to be suitable for non-invasive or minimally invasive surgical and medical applications, such as being arrangeable within an endoscope or a laparoscope.

Furthermore, the pads or cushions 60 which may be mounted between the two jaws 12, 14 on their facing surfaces may be removable and replaceable in accordance with the particular needs and requirements for the clamping devices, and also for replacement thereof prior to sterilizing of the device or clamp for repeated use with the same or other patients.

In one embodiment, as described above, clamp head 10 is sized and configured to pass through a 10 mm trocar or opening. As a result, the surgical clamp of this invention may be used to clamp blood vessels, for example, in a minimally invasive surgical procedure that utilizes small openings in a patient's body through which to insert instruments. Such an opening can be located between the ribs in an intercostal space.

In a minimally invasive procedure, clamp head 10 is initially configured to be positioned in a first position, such that jaws 12, 14 are relatively close together or contacting one another. A small opening is formed in a patient's body and clamp head 10 is passed through the small opening. Clamp head 10 is then positioned near a vessel, such as the aorta, and the user operates an operative mechanism, such as cable 16, of the clamp to drive the actuation structure, for example, linkage mechanism 18, to move jaws 12, 14 to a second position, where jaws 12, 14 are spaced apart in parallel a distance greater than that of the first position. Cable 16 may be actuated from a position outside the patient's body by any actuator known to those skilled in the art.

In the event that the distance between jaws 12, 14 is not large enough to accommodate the diameter of the blood vessel the user intends to clamp, the user may further operate the operative mechanism to move the actuation structure such that jaws 12, 14 assume a third position. In the third position, the distal end of at least one of the jaws has pivoted about a point proximal to the distal end such that the distal ends of jaws 12, 14 are spaced apart a distance greater than the distance that separated the distal ends of jaws 12, 14 when they were in the second position. Of course, the user can move the jaws directly from the first position to the third position. The user can then position jaws 12, 14 about the blood vessel, and clamp the vessel by appropriately operating the operative mechanism. As jaws 12, 14 move closer together, the actuation structure ensures that jaws 12, 14 first move relative to one another from an open, third position to a parallel, second position, and then to a closed, parallel first position, where the blood vessel is at least substantially occluded. In this manner, the blood vessel is initially grasped by using scissors-type motion, and is thereafter occluded by using a parallel motion to substantially proportionately compress the tissue of the blood vessel between jaws 12, 14. The clamp can be locked in the closed position by any means known to one skilled in the art.

Once the surgical procedure is complete, the user reverses the above steps. First jaws 12, 14 are opened to permit blood to flow through the blood vessel by operating the operative mechanism, which moves jaws 12, 14 from the first position to the second or third position, as appropriate. Clamp head 10 is repositioned such that jaws 12, 14 are not disposed about the blood vessel. Jaws 12, 14 are once again moved to the first position to minimize the profile of clamp head 10, so that clamp head 10 can be passed out of the opening in the patient's body.

Of course, while the clamp is described above as being used in a minimally invasive procedure, it can just as easily be used in an open surgical procedure. Cable 16 can be detachably connected to a handle or actuator of some sort such that the handle can be attached and detached when the clamp head is located within the patient's body. Further, cable 16 can be designed to be detachably connected to clamp head 10 such that clamp head 10 can be inserted into the patient's body via one opening, and cable 16 is introduced through a separate opening. In this way, clamp head 10 and cable 16 can be attached while clamp head 10 is in the body. In addition, although described with regard to the use of the clamping of various body vessels, and particularly the aorta, the clamping devices and their methods of use are naturally also applicable to numerous other physical applications and locations within the body of a patient.

While there has been shown and described what are considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

1. A surgical clamp, comprising:
a clamp head;
a first jaw and a second jaw, each mounted to the clamp head for movement toward and away from each other;
an operative mechanism coupled to the proximal end of at least one of the first jaw and the second jaw; and
an actuating structure connected to the operative mechanism for imparting a parallel opening movement to at least one of the first jaw and the second jaw from a first position to a second position where the first jaw and the second jaw are spaced apart and parallel to one another, the actuating structure selectively imparting further scissors-like opening movement to at least one of the first jaw and the second jaw from the second position to a third position where the first jaw and the second jaw are spaced apart a distance greater than that of the second position.

2. The surgical clamp of claim 1, wherein the other of the first jaw and the second jaw is maintained in a fixed position relative to the clamp head during the opening movement between the first and second jaws.

3. The surgical clamp of claim 1, wherein the first jaw includes a plate structure at the proximal end, the plate structure having an elongate slot formed therein; the second jaw including a plate structure at the proximal end having an elongate slot formed therein, the slots extending in parallel spaced relationships; and the operative mechanism comprises a linkage arrangement having first and second guide pins extending into respectively each of the elongate slots, whereby the actuating structure imparts opening movement to the linkage arrangement by slidably displacing the guide pins in the slots to form the parallel spacing between the jaws.

4. The surgical clamp of claim 3, wherein the slot in the plate structure of the first jaw includes an angled slot portion, whereby upon further actuation of the operative mechanism, the first guide pin is displaced into the angled slot portion causing the linkage arrangement to pivot the first jaw into a scissors-like wider opening between the jaws.

5. The surgical clamp of claim 3, wherein the linkage arrangement comprises a plurality of closeable and openable parallel scissors links.

6. The surgical clamp of claim 4, wherein the first and second jaws of the pair of jaws are simultaneously openable and closeable responsive to actuation of the operative mechanism.

7. The surgical clamp of claim 6, wherein opening movement of the second jaw is provided by a guide pin slidably arranged in a link member of the linkage system.

8. The surgical clamp of claim 1, wherein the clamp head comprises an angled linkage member pivotably attached to the proximal ends of the first and second elongate jaws, the first jaw including a plate structure having a vertically extending slot with an angled upper slot portion; a guide pin at the upper end of the angled linkage member being slidable within the slot whereby upon actuation of the linkage member by the actuating structure the guide pin is displaced upwardly in the slot so as to initially open the first jaw in parallel relationship with the second jaw, and upon the guide pin entering the upper angled slot portion further pivoting the second jaw in a scissors-like wider opening displacement.

9. The surgical clamp of claim 1, wherein elastomeric cushioning means are provided on the facing surfaces of the first and second elongated jaws.

10. The surgical clamp of claim 1, wherein the first and second jaws are curved along the axial lengths thereof to accommodate the curvature of body vessels.

11. The surgical clamp of claim 1, wherein the actuating structure comprises a cable actuatable by extending through an endoscopic or laparoscopic device.

12. The surgical clamp of claim 1, wherein the first jaw and the second jaw are dimensioned to provide an operative length of about 65-75 mm.

13. The surgical clamp of claim 12, wherein the first jaw and the second jaw are spaced apart about 10-12 mm when in the first position, and are spaced apart up to about 40 mm when in the third position.

14. A clamp, comprising:
a first jaw including a first slot extending along a first plane;
a second jaw including a second slot extending along a second plane, the first and second jaws each being arranged such that the first and second planes are substantially parallel to each other, one of the first and second slots having an angled slot portion extending along a third plane at an angle to the first and second planes;
a linkage mechanism having at least a first pin for engagement in the first slot and at least a second pin for engagement in the second slot; and
an actuator for displacing the linkage mechanism from a first position, wherein the first pin and the second pin are positioned in the first and second slots in a substantially parallel configuration, to a second position, wherein one of the first pin and the second pin is positioned within the angled slot portion.

15. The clamp of claim 14, wherein the linkage mechanism comprises a plurality of closeable and openable parallel scissors links.

16. The clamp of claim 14, wherein the first and second jaws are simultaneously openable and closeable responsive to actuation of the linkage mechanism.

17. The clamp of claim 15, wherein opening movement of the second jaw is provided by a guide pin slidably arranged in a link member of the linkage system.

18. The clamp of claim 14, wherein the actuator comprises a cable.

19. The clamp of claim 14, comprising elastomeric cushioning means are provided on the facing surfaces of the first and second elongated jaws.

20. The clamp of claim 19, wherein the elastomeric cushioning means comprise replaceable resilient pads mounted on the jaws, the pads being formed of a fabric or plastic material.

21. A method of occluding a body vessel with a surgical clamp including a clamp head mounting a pair of elongate jaws for movement toward and away from each other, the method comprising:
coupling an operative mechanism to proximal ends of each the jaws; and
connecting an actuating structure to the operative mechanism for imparting parallel opening movement to at least a first one of the pair of jaws from a closed position into a parallel spaced position with a second the jaw, and causing the actuating structure to selectively impart further scissors-like opening movement to the at least first one of the pair of jaws into a wider open position between the jaws.

22. The method of claim 21, wherein the second the jaw of the pair of jaws is maintained in a fixed position with the clamp head during the opening movement between the first and second jaws.

23. The method of claim 21, wherein the parallel spaced motion between the first and second jaws is within a range of about 10-12 mm, and the further scissors-like motion provides for an opening of up to about 40 mm between the jaws.

24. A method of occluding a blood vessel with a clamp, comprising the steps of:
providing a clamp head, comprising:
a first jaw and a second jaw, each mounted to the clamp head for movement toward and away from each other;
an operative mechanism coupled to the proximal end of at least one the first jaw and the second jaw; and
an actuating structure connected to the operative mechanism for imparting parallel opening movement to at least one of the first jaw and the second jaw from a first position to a second position where the first jaw and the second jaw are spaced apart and parallel to one another, the actuating structure selectively imparting further scissors-like opening movement to at least one of the first jaw and the second jaw from the second position to a third position where the first jaw and the second jaw are spaced apart a distance greater than that of the second position;
creating an opening in a patient's body;
positioning the first jaw and the second jaw in the first position;
passing the clamp head through the opening;
actuating the actuating structure to cause one of the first jaw and the second jaw to move to the third position;
positioning the first jaw and the second jaw such that the blood vessel is disposed between first jaw and the second jaw; and
actuating the actuating structure to cause one of the first jaw and the second jaw to move to the first position to clamp the blood vessel.

25. The method of claim 24, comprising the steps of:
actuating the actuating structure to cause one of the first jaw and the second jaw to move to the second position prior to clamping the blood vessel.

26. The method of claim 24, wherein the opening is a small opening.

27. The method of claim 24, wherein the opening is an intercostal opening.

28. The method of claim 24, wherein the clamp head is passed through a trocar positioned within the opening.
